(19) United States
(12) Patent Application Publication
(10) Pub. No.: US 2013/0137134 A1
(43) Pub. Date: May 30, 2013

(54) METHOD AND SYSTEM FOR DETECTING AND MONITORING HEMATOLOGICAL CANCER

(71) Applicants: Shaul Mordechai, Omer (IL); Joseph Kapelushnik, Neve Ilan (IL); Ilana Nathan, Omer (IL); Udi Zelig, D.N. HaNegev (IL)

(72) Inventors: Shaul Mordechai, Omer (IL); Joseph Kapelushnik, Neve Ilan (IL); Ilana Nathan, Omer (IL); Udi Zelig, D.N. HaNegev (IL)

(73) Assignee: BEN GURION UNIVERSITY OF THE NEGEV RESEARCH AND DEVELOPMENT AUTHORITY, Beer Sheva (IL)

(21) Appl. No.: 13/638,367

(22) PCT Filed: Mar. 29, 2011
(86) PCT No.: PCT/IL2011/000282

§ 371 Date: Dec. 17, 2012

Related U.S. Application Data
(60) Provisional application No. 61/318,395, filed on Mar. 29, 2010.

(51) Int. Cl. C12Q 1/04; C12Q 1/02

(52) U.S. Cl. 435/34

(57) ABSTRACT

A method for diagnosis of a hematological malignancy of a subject is provided. The method comprises obtaining a second derivative of an infrared (IR) spectrum of a population of mononuclear cells by analyzing the population of mononuclear cells by infrared spectroscopy, and based on the second derivative of the infrared spectrum, generating an output indicative of the presence of a hematological malignancy. Other applications are also described.

### CROSS-REFERENCES TO RELATED APPLICATIONS

The present application claims the priority of U.S. Provisional Application 61/318,395 to Mordechai et al., filed Mar. 29, 2010, which is incorporated herein by reference.

### FIELD OF EMBODIMENTS OF THE INVENTION

Applications of the present invention relate generally to diagnosis and monitoring of cancer, and particularly to methods for diagnosis and monitoring of hematological neoplasms.

### BACKGROUND

Hematological malignancies are the types of cancer that affect blood, bone marrow, and lymph nodes.

Acute leukemia is a common neoplasia in children and adolescents and is characterized by a rapid increase in the numbers of immature blood cells in the bone marrow, blood, and other tissues. In the last few decades, there has been an advance in the development of antileukemic agents and treatment protocols, which have led to a cure rate of above 80% of acute lymphoblastic leukemia in children and adolescents [Pui 2006, Tucci 2008].

Clinical studies point to the complexity in determining the risk level and administration of the optimal protocol for every individual patient [Vrooman 2009]. Currently, leukemia prognosis includes several parameters such as age, leukocytes count, immunophenotyping, and blasts presence in the peripheral blood (PB) and bone marrow (BM) at the 7th day and other days along the treatment [Tucci 2008, Smith 1996, Campana 2008]. To evaluate patients' response, minimal residual disease (MRD) is determined either by polymerase chain reaction (PCR) or by flow cytometry (FACS) measurements of blasts in the bone marrow [Vrooman 2009, Campana 2008, Cazzaniga 2005].

Fourier Transform Infrared (FTIR) spectroscopy is used to identify biochemical compounds and examine the biochemical composition of a biological sample. FTIR spectroscopy is typically a simple, reagent-free and rapid method which offers information regarding macromolecular structure and composition of biological sample. Typically, FTIR spectra are composed of several absorption bands, each corresponding to specific functional groups related to cellular components such as lipids, proteins, carbohydrates and nucleic acids. Processes such as carcinogenesis may trigger global changes in cellular biochemistry, resulting in differences in the absorption spectra when analyzed by FTIR spectroscopy techniques. Therefore, FTIR spectroscopy is commonly used to distinguish between normal and abnormal tissue by analyzing the changes in absorption bands of macromolecules such as lipids, proteins, carbohydrates and nucleic acids. Additionally, FTIR spectroscopy may be utilized for evaluation of cell death mode, cell cycle progression and the degree of maturation of hematopoietic cells. [Diem 2008, Diem 2004, Liu K Z 2007, Sahu 2005, Sahu 2006, Zelig 2009, Boydston-White 1999].

The following references may be of interest:

Agatha G., et al., Fatty acid composition of lymphocyte membrane phospholipids in children with acute leukemia. Cancer Lett. 2001 Nov. 28; 173(2):139-44.

Andrus PG. Cancer monitoring by FTIR spectroscopy. Technol Cancer Res Treat. 2006 April; 5(2):157-67.

Basso G, et al., Risk of relapse of childhood acute lymphoblastic leukemia is predicted by flow cytometric measurement of residual disease on day 15 bone marrow. J Clin Oncol. 2009 Nov. 1; 27(31):5168-74.

Bogomolny E., et al., Early spectral changes of cellular malignant transformation using Fourier transformation infrared microspectroscopy. 2007. J Biomed Opt. 12:024003

Boydston-White S T., et al., Infrared spectroscopy of human tissue V infrared spectroscopic studies of myeloid leukemia (ML-1) cells at different phases of cell cycle. Biospectroscopy 1999 5:219-227.

Campana D. Molecular determinants of treatment response in acute lymphoblastic leukemia. Hematology Am Soc Hematol Educ Program. 2008:366-73.

Castillo L. A Randomized Trial of the I-BFM-SG for the Management of Childhood non-B Acute Lymphoblastic Leukemia. ALL IC-BFM 2002.

Cazzaniga G, Biondi A. Molecular monitoring of childhood acute lymphoblastic leukemia using antigen receptor gene rearrangements and quantitative polymerase chain reaction technology. Haematologica. 2005 March; 90(3):382-90.

Diem M., et al., A decade of vibrational micro-spectroscopy of human cells and tissue (1994-2004). Analyst 129, 88-885 (2004)

Diem M., et al., Vibrational spectroscopy for medical diagnosis. John Wiley & Sons. New York, 2008

Everitt B., Cluster Analysis, John Wiley and Sons, New York (1980).

Gottfried E L., Lipids of human leukocytes: relation to celltype. J. Lipid. Res. 1967 July; 8(4):321-7.

Hengartner, M. O. The biochemistry of apoptosis. Nature. 2000, 407: 770-776.

Hildebrand J., et al., Neutral glycolipids in leukemic and nonleukemic leukocytes. J Lipid Res. 1971 May; 12(3):361-6.

Hoffman, R., et al., Hematology-Basic Principles and Practice, 3rd Edition 2000.

Hudson L, Poplack F C. Practical immunology. Blackwell Publication: London, 1976.

Inbar M., et al., Cholesterol as a bioregulator in the development and inhibition of leukemia. Proc Natl Acad Sci USA. 1974 October; 71(10):4229-31.

Inbar M., et al., Fluidity difference of membrane lipids in human normal and leukemic lymphocytes as controlled by serum components. Cancer Res. 1977 September; 37(9):3037-41.

Krishna CM., et al., Combined Fourier transform infrared and Raman spectroscopic approach for identification of multidrug resistance phenotype in cancer cell lines. Biopolymers. 2006 Aug. 5; 82(5):462-70.

Lavie Y, et al., Changes in membrane microdomains and caveolae constituents in multidrug-resistant cancer cells. Lipids. 1999; 34 Suppl: S57-63.

Liu KZ., et al., Bimolecular characterization of leucocytes by infrared spectroscopy. Br J Haematol. 2007 March; 136 (5):713-22

Mantsch M and Chapman D. Infrared spectroscopy of bio molecules. John. Wiley New York 1996

Mihaela O and Pui C H, Diagnosis and classification, in Childhood Leukemias 2nd ed. edited by C. H. Pui (Cambridge: Cambridge University Press, 2006), pp. 21-47.

Pui C H, Evans W E. Treatment of acute lymphoblastic leukemia. N Engl J Med 2006; 354: 166-78.

Sahu R K., et al., Continuous monitoring of WBC (biochemistry) in an adult leukemia patient using advanced FTIR-spectroscopy. Leuk Res. 2006 June; 30(6):687-93.

Sahu R K., et al., Can Fourier transform infrared spectroscopy at higher wavenumbers (mid IR) shed light on biomarkers for carcinogenesis in tissues? J. Biomed Opt. 2005 September-October; 10(5):054017.

Smith M, et al., Uniform approach to risk classification and treatment assignment for children with acute lymphoblastic leukemia. J Clin Oncol 1996; 14: 18-24.

Spiegel, R J., et al., Plasma lipids alterations in leukemia and lymphoma. Am. T. Med. 1982. 72: 775-781.

Toyran N., et al., Selenium alters the lipid content and protein profile of rat heart: an FTIR microspectroscopy study. Arch. Biochem. Biophys. 458:184-193.

Tucci F, Aricò M. Treatment of pediatric acute lymphoblastic leukemia. Haematologica. August; 93(8):1124-8.

Vrooman L M, Silverman L B. Childhood acute lymphoblastic leukemia: update on prognostic factors. Curr Opin Pediatr. 2009 February; 21(1):1-8.

Zelig U., et al., Diagnosis of cell death by means of infrared spectroscopy. Biophys J 2009 Oct. 7; 79:2107-14.

### SUMMARY OF APPLICATIONS OF THE INVENTION

In some applications of the present invention, infrared (IR) spectroscopy, e.g., Fourier transform infrared (FTIR) spectroscopy and microspectroscopy (FTIR-MSP), is utilized for detecting and/or monitoring a hematological cancer such as, but not limited to, leukemia.

In some applications of the present invention, a method is provided for the diagnosis of multiple types of hematological neoplasms, e.g., various types of leukemia. Typically, the method comprises analysis by infrared (IR) spectroscopy, of global biochemical properties of blood-derived mononuclear cells for the detection of a hematological cancer.

In accordance with some applications of the present invention, blood-derived mononuclear cells from leukemia patients are analyzed by FTIR microspectroscopy techniques. The FTIR spectra of the mononuclear cells of the leukemia patients are compared to the FTIR spectra of mononuclear cells of healthy controls and subjects suffering from clinical symptoms that are similar to leukemia e.g., fever.

For some applications, a data processor is configured to calculate a second derivative of an infrared (IR) spectrum (e.g., a second derivative of an FTIR spectrum) of the mononuclear cells and, based on the second derivative of the infrared (IR) spectrum, to generate an output indicative of the presence of a hematological malignancy.

The inventors have identified that the mononuclear cell samples obtained from leukemia patients produce FTIR spectra that differ from those of the healthy controls and the non-cancer patients suffering from clinical symptoms that are similar to leukemia, e.g., subjects with a fever, thereby allowing differential diagnosis of the leukemia patients. By distinguishing among the leukemia patients, patients with clinical symptoms that are similar to leukemia, and healthy controls, IR spectroscopy provides an effective diagnostic tool for diagnosis of leukemia and/or other types of hematological malignancies.

Additionally or alternatively, some methods of the present invention are used to provide monitoring and follow up of hematological cancer patients during and after treatment such as, but not limited to, chemotherapy treatment. Typically, changes in FTIR spectra of mononuclear cells of leukemia patients who are undergoing treatment can indicate biochemical changes in the cells in response to the treatment. This biochemical information can contribute to establishing a prognosis as well as providing insight into the effect of treatment on the patient and/or the malignancy.

There is therefore provided in accordance with some applications of the present invention a method for diagnosis of a hematological malignancy of a subject, the method including:

obtaining a second derivative of an infrared (IR) spectrum of a population of mononuclear cells by analyzing the population of mononuclear cells by infrared spectroscopy; and

based on the second derivative of the infrared spectrum, generating an output indicative of the presence of a hematological malignancy.

For some applications, analyzing the cells by infrared (IR) spectroscopy includes analyzing the cells by Fourier Transformed Infrared (FTIR) spectroscopy.

For some applications, analyzing the cells by infrared (IR) spectroscopy includes analyzing the cells by Fourier Transformed Infrared microspectroscopy (FTIR-MSP).

For some applications, analyzing includes assessing a characteristic of the mononuclear cell sample at a wavenumber of 2853±4 cm-1.

For some applications, analyzing includes assessing a characteristic of the mononuclear cell sample at a wavenumber of 967±4 cm-1.

For some applications, analyzing includes assessing a characteristic of the mononuclear cell sample at at least one wavenumber selected from the group consisting of: 2923±4, 1625±4, 1313±4, 1172±4, 1155±4, 1085±4, 1052±4, 780±4 and 740±4 cm-1.

For some applications, analyzing includes assessing the characteristic at at least two wavenumbers selected from the group.

For some applications, analyzing includes assessing the characteristic at at least three wavenumbers selected from the group.

For some applications, the hematological malignancy includes leukemia, and generating the output includes generating an output indicative of the presence of leukemia.

For some applications, the leukemia includes a type of leukemia selected from the group consisting of: acute lymphoblastic leukemia (ALL) and acute myeloblastic leukemia (AML), and generating the output includes generating an output indicative of a type of leukemia selected from the group.

There is further provided, in accordance with some applications of the present invention, a method for diagnosis of a hematological malignancy of a subject, the method including:

obtaining an infrared (IR) spectrum of a population of mononuclear cells by analyzing the population of mononuclear cells by infrared (IR) spectroscopy; and

based on one or more individual bands of the infrared spectrum, generating an output indicative of the presence of a hematological malignancy, without calculating a ratio between two of the bands.

For some applications, generating the output includes generating the output without calculating any relationship relating individual ones of the bands.

For some applications, analyzing the cells by infrared (IR) spectroscopy includes analyzing the cells by Fourier Transformed Infrared (FTIR) spectroscopy.

For some applications, analyzing the cells by infrared (IR) spectroscopy includes analyzing the cells by Fourier Transformed Infrared microspectroscopy (FTIR-MSP).

For some applications, analyzing includes assessing a characteristic of the mononuclear cell sample at a wavenumber of 967±4 cm-1.

For some applications, analyzing includes assessing a characteristic of the mononuclear cell sample at a wavenumber of 2853±4 cm-1.

For some applications, analyzing includes assessing a characteristic of the mononuclear cell sample at at least one wavenumber selected from the group consisting of: 2923±4, 1625±4, 1313±4, 1172±4, 1155±4, 1085±4, 1052±4, 780±4 and 740±4 cm-1.

For some applications, analyzing includes assessing the characteristic at at least two wavenumbers selected from the group.

For some applications, analyzing includes assessing the characteristic at at least three wavenumbers selected from the group.

There is still further provided, in accordance with some applications of the present invention a method for monitoring the effect of an anti-cancer treatment on a subject undergoing anti-cancer treatment for a hematological malignancy, for use with a first population of mononuclear cells obtained from the subject prior to initiation of the treatment and a second population of mononuclear cells obtained from the subject after initiation of the treatment, the method including:

obtaining respective second derivatives of infrared (IR) spectra of the first and second populations of mononuclear cells, by analyzing the first and second populations of mononuclear cells by IR spectroscopy; and

based on the second derivatives of the IR spectra, generating an output indicative of the effect of the treatment.

For some applications the method includes, obtaining an IR spectrum of a third population of mononuclear cells obtained from the subject following termination of the treatment, by analyzing the third population of mononuclear cells by IR spectroscopy.

For some applications, generating the output includes generating the output without calculating any relationship relating individual ones of the bands.

For some applications, analyzing the cells by IR spectroscopy includes analyzing the cells by Fourier Transformed infrared spectroscopy.

For some applications, analyzing the cells by infrared spectroscopy includes analyzing the cells by Fourier Transformed Infrared microspectroscopy (FTIR-MSP).

For some applications, analyzing includes assessing a characteristic of the mononuclear cell sample at a wavenumber of 967±4 cm-1.

For some applications, analyzing includes assessing a characteristic of the mononuclear cell sample at a wavenumber of 2853±4 cm-1.

For some applications, analyzing includes assessing a characteristic of the mononuclear cell sample at at least one wavenumber selected from the group consisting of: 2923±4, 1625±4, 1313±4, 1172±4, 1155±4, 1085±4, 1052±4, 780±4 and 740±4 cm⁻¹.

For some applications, analyzing includes assessing the characteristic at at least two wavenumbers selected from the group.

For some applications, analyzing includes assessing the characteristic at at least three wavenumbers selected from the group.

For some applications, the effect of the treatment includes an effect selected from the group consisting of: a good response, an intermediate response, an unfavorable response, remission, and relapse; and

generating the output indicative of the effect of the treatment includes generating the output indicative of the effect selected from the group.

There is additionally provided, in accordance with some applications of the present invention a method for detecting a hematological malignancy of a subject, the method including:

obtaining a second derivative of an infrared (IR) spectrum of a population of white blood cells by analyzing the population of white blood cells by IR spectroscopy; and

based on the second derivative of the IR spectrum, generating an output indicative of the presence of a hematological malignancy.

For some applications, analyzing the cells by IR spectroscopy includes analyzing the cells by Fourier Transformed Infrared spectroscopy.

For some applications, analyzing the cells by infrared spectroscopy includes analyzing the cells by Fourier Transformed Infrared microspectroscopy (FTIR-MSP).

There is yet additionally provided, in accordance with some applications of the present invention, a method for diagnosis of a hematological malignancy, the method including:

obtaining an infrared (IR) spectrum of a population of mononuclear cells obtained from a subject suffering from a clinical symptom of a hematological malignancy, by analyzing the cells by infrared spectroscopy; and

based on the infrared (IR) spectrum, generating an output that indicates that it is differentially indicative of the presence of a hematological malignancy versus the presence of a symptom selected from the group consisting of fever and elevated white blood cell (WBC) count.

There is yet further provided, in accordance with some applications of the present invention, a system for diagnosing a hematological malignancy, including a data processor configured to calculate a second derivative of an infrared (IR) spectrum of mononuclear cells of a subject and, based on the second derivative of the infrared (IR) spectrum, to generate an output indicative of the presence of a hematological malignancy.

For some applications, the IR spectrum includes a Fourier Transformed Infrared (FTIR) spectrum, and the data processor is configured to calculate a second derivative of the FTIR spectrum.

For some applications, the hematological malignancy includes leukemia, and the data processor is configured to generate an output indicative of the presence of leukemia.

For some applications, the leukemia includes a type of leukemia selected from the group consisting of: acute lymphoblastic leukemia (ALL) and acute myeloblastic leukemia (AML), and the data processor is configured to generate an output indicative of the presence of a type of leukemia selected from the group.

There is additionally provided, in accordance with some applications of the present invention, a system for monitoring the effect of an anti-cancer treatment on a subject undergoing anti-cancer treatment for a hematological malignancy, the system including a data processor configured to calculate a second derivative of an infrared (IR) spectrum of mononuclear cells of a subject and, based on the second derivative of the infrared (IR) spectrum, to generate an output indicative of the effect of the treatment.

For some applications, the IR spectrum includes a Fourier Transformed Infrared (FTIR) spectrum, and the data processor is configured to calculate a second derivative of the FTIR spectrum.

For some applications, the effect of the treatment includes an effect selected from the group consisting of: a good response, an intermediate response, an unfavorable response, remission, and relapse; and

the data processor is configured to generate the output indicative of the effect of the treatment selected from the group.

The present invention will be more fully understood from the following detailed description of embodiments thereof, taken together with the drawings, in which:

### BRIEF DESCRIPTION OF THE DRAWINGS

FIGS. 1A-B are graphs representing IR absorption spectra and the second derivative of the IR spectra of mononuclear cells of leukemia patients, fever patients, and healthy controls, derived in accordance with some applications of the present invention;

FIGS. 2A-D are graphs showing spectral analysis of specific IR absorption bands used for leukemia diagnosis and cluster analysis thereof, derived in accordance with some applications of the present invention;

FIGS. 3A-C are graphs representing FTIR microspectroscopy spectral analysis of mononuclear cells from peripheral blood (PB), and flow cytometry analysis of bone marrow (BM) samples of a first selected leukemia patient during the treatment, derived in accordance with some applications of the present invention;

FIGS. 4A-D are graphs representing FTIR microspectroscopy spectral analysis of mononuclear cells from peripheral blood (PB), and flow cytometry analysis of bone marrow (BM) samples of a second selected leukemia patient during the treatment, derived in accordance with some applications of the present invention;

FIGS. 5A-C are graphs representing FTIR microspectroscopy spectral analysis of mononuclear cells from peripheral blood (PB), and flow cytometry analysis of bone marrow (BM) samples of a third selected leukemia patient during the treatment, derived in accordance with some applications of the present invention; and

FIGS. 6A-B are graphs representing FTIR microspectroscopy spectral analysis of mononuclear cells from peripheral blood (PB), and flow cytometry analysis of bone marrow (BM) samples of five additional selected leukemia patient during the treatment, derived in accordance with some applications of the present invention.

### DETAILED DESCRIPTION OF THE EMBODIMENTS

Some applications of the present invention comprise diagnosis of a hematological malignancy by IR spectroscopy, e.g., FTIR microspectroscopy (FTIR-MSP) techniques. Some applications of the present invention comprise obtaining a blood sample from a subject and analyzing mononuclear cells from the sample by FTIR-MSP techniques for the detection of a hematological malignancy. Typically, the Peripheral Blood Mononuclear Cells (PBMC) of a patient suffering from a hematological cancer are identified as exhibiting FTIR spectra that are different from FTIR spectra produced by mononuclear cells from a healthy subject and from a subject suffering from clinical symptoms similar to those of a hematological cancer, e.g., a fever. Accordingly, some applications of the present invention provide a useful method for the diagnosis of hematological cancer. Generally, FTIR spectra of mononuclear cells obtained from a hematological cancer patient reflect biochemical changes which occur in those cells.

In addition, some applications of the present invention are useful for supplying biochemical information at the molecular level regarding the response of a leukemia patient to treatment, particularly, but not exclusively, chemotherapy treatment. A long term follow-up of leukemia patients using FTIR-MSP was conducted as described herein below. The spectral results were typically analyzed in parallel with the routine tests of blasts presence in the bone marrow (BM), to evaluate the patients' response to chemotherapy, determined by flow cytometry.

In accordance with some applications, mononuclear cells are isolated from the peripheral blood and subjected to IR spectroscopy, e.g., FTIR-MSP. Reduced lipids, elevated DNA absorptions and other characteristic spectral bands are then used as parameters for diagnosis of hematological cancer, such as, but not limited to, leukemia. In various exemplary applications of the invention, one or more of the following wavenumbers are utilized for the detection and monitoring of a hematological cancer: 2923±4, 2854±4, 1625±4, 1313±4, 1172±4, 1155±4, 1085±4, 1052±4, 967±4, 780±4 and 740±4 cm-1. Other spectral bands and their corresponding functional groups in the cell are provided in Table II, below. In some applications as described hereinbelow, in order to increase accuracy, a second derivative of vector-normalized spectra is used. It is to be understood that any normalization technique or spectral manipulation that utilizes the above spectral bands including, without limitation, 966/amide II or CH2/CH3 at 2835-3000 cm-1, is included in the scope of the present invention (optionally in combination with one or more other spectral bands).

Representative examples for a hematological cancer include, without limitation, acute lymphoblastic leukemia (ALL), acute lymphoblastic β-cell leukemia, acute lymphoblastic T-cell leukemia, acute nonlymphoblastic leukemia (ANLL), acute myeloblastic leukemia (AML), acute promyelocytic leukemia (APL), acute monoblastic leukemia, acute erythroleukemic leukemia, acute megakaryoblastic leukemia, chronic myelocytic leukemia (CML), chronic lymphocytic leukemia (CLL), multiple myeloma, myelodysplastic syndrome (MDS), and chronic myelo-monocytic leukemia (CMML), wherein MDS may be either refractory anemia with excessive blast (RAEB) or RAEB in transformation to leukemia (RAEB-T).

### Methods Used in Some Embodiments of the Present Invention

A series of protocols are described hereinbelow which may be used separately or in combination, as appropriate, in accordance with applications of the present invention. It is to be appreciated that numerical values are provided by way of illustration and not limitation. Typically, but not necessarily, each value shown is an example selected from a range of values that is within 20% of the value shown. Similarly, although certain steps are described with a high level of specificity, a person of ordinary skill in the art will appreciate that other steps may be performed, mutatis mutandis.

In accordance with some applications of the present invention, the following methods were applied:

### Obtaining Patient and Control Populations

All studies were approved by the Ethics Committee of the Soroka University Medical Center and conducted in accordance with the Declaration of Helsinki. Qualified personnel obtained informed consent from each parent of an individual patient participating in this study.

The patient population included 15 patients with a variety of leukemia types. The patients were treated according to IC-BFM 2002 protocol [ALL IC-BFM 2002]. Patient data are described in Table I below:

TABLE I

| | | WBC | Blasts in | Blasts in | | |
|---|---|---|---|---|---|---|
| Patient | Age | count | PB (%) | BM (%) | Diagnosis | Prognosis* |
| 1 | 3 | 10.44 | 40 | 90 | Early B | Good |
| | | | | | ALL | |
| 2 | 10 | 3.9 | 1 | 50 | AML-M0 | Unfavorable |
| 3 | 2 | 7.6 | 13 | 80 | Pre B ALL | Good |
| 4 | 1 | 10.2 | 25 | 90 | Pre B ALL | Good |
| 5 | 5 | 261 | 56 | 95 | T Cell ALL | Intermediate |
| 6 | 4 | 16.8 | — | 90 | Pre B ALL | Good |
| 7 | 14 | 19.57 | 90 | 95 | Pre B ALL | Unfavorable |
| 8 | 17 | 82.95 | 71 | 95 | AML-M1 | Unfavorable |
| 9 | 2 | 24.52 | 0 | 80 | CALLA + | Good |
| | | | | | ALL | |
| 10 | 7 | 624.2 | 96 | 95 | Pre B ALL | Unfavorable |
| 11 | 17 | 3.8 | 0 | 80 | Pre B ALL | Unfavorable |
| 12 | 6 | 11.08 | — | — | Pre B ALL | Unfavorable |
| 13 | 2 | 3 | 38 | 90 | Pre B ALL | Good |
| 14 | 1 | 379.4 | 92 | 95 | Pre B ALL | Unfavorable |
| 15 | 3 | 1.8 | 0.04 | 90 | Early B | Good |
| | | | | | ALL | |

*Good prognosis: Ages 2-6, WBC <20,000, Philadelphia-negative clone, Blast <1000 in PB at day 7, Blast <0.1% in BM at day 33.
Unfavorable prognosis: Relapse, Philadelphia-positive clone, Blasts >1000 in PB at day 7, Blast >0.1% in BM at day 33.

The non-cancer group exhibiting clinical symptoms similar to a hematological cancer (n=19) were diagnosed with high fever and/or a high white blood cell (WBC) count.

The control group (n=27) included healthy volunteers who underwent detailed clinical questioning, at the Soroka University Medical Center and Ben-Gurion University.

### Collection of Blood Samples

1-2 ml of peripheral blood was collected in 5 ml EDTA blood collection tubes from leukemia patients, subjects with fever and/or a high white blood cell (WBC) count, and healthy controls, using standardized phlebotomy procedures. Samples were processed within 1-2 hours of collection.

### Isolation of Peripheral Blood Mononuclear Cells (PBMC)

Platelet-depleted residual leukocytes obtained from cancer patients, subjects with fever and/or a high white blood cell (WBC) count, and healthy controls were applied to Histopaque 1077 gradients (Sigma Chemical Co., St. Louis, Mo., USA) following the manufacturer's protocol to obtain PBMC.

The cells were aspirated from the interface, washed twice with isotonic saline (0.9% NaCl solution) at 250 g, and resuspended in 5 μl fresh isotonic saline. 1.5 μl of washed cells were deposited on zinc selenide (ZnSe) slides to form approximately a monolayer of cells, and then air dried for 1 h under laminar flow to remove water. The dried cells were then measured by FTIR microspectroscopy.

### FTIR-Microspectroscopy

Fourier Transform Infrared Microspectroscopy (FTIR-MSP) and Data Acquisition Measurements on cell cultures were performed using the FTIR microscope IR scope 2 with a liquid-nitrogen-cooled mercury-cadmium-telluride (MCT) detector, coupled to the FTIR spectrometer Bruker Equinox model 55/S, using OPUS software (Broker Optik GmbH, Ettlingen, Germany). To achieve high signal-to-noise ratio (SNR), 128 coadded scans were collected in each measurement in the wavenumber region 700 to 4000 cm-1. The measurement site was circular with a diameter of 100 μm and a spectral resolution of 4 cm-1. To reduce cell amount variation and guarantee proper comparison between different samples, the following procedures were adopted:

1. Each sample was measured at least five times at different spots.
2. ADC rates were empirically chosen between 2000 to 3000 counts/sec (providing measurement areas with similar cellular density).
3. The obtained spectra were baseline corrected using the rubber band method, with 64 consecutive points and normalized using vector normalization in OPUS software as described in an article by Bogomolny E., et al., entitled: Early spectral changes of cellular malignant transformation using Fourier transformation infrared microspectroscopy. 2007. J Biomed Opt. 12:024003.

In order to obtain precise absorption values at a given wavenumber with minimal background interference, the second derivative spectra were used to determine concentrations of bio-molecules of interest. The value of the maxima was subtracted from the minima in the second derivative spectra for each band. This value is equivalent to evaluating the band value from the peak to the base of the band in the raw spectra. This method is susceptible to changes in FWHM (full width at half maximum) of the IR bands. However, in the case of biological samples, all cells from the same type are composed from similar basic components which give relatively broad bands. Thus, it is possible to generally neglect the changes in bands FWHM, as described in an article by Toyran N., et al., entitled: Selenium alters the lipid content and protein profile of rat heart: an FTIR microspectroscopy study. Arch. Biochem. Biophys. 458:184-193.

### Statistical Analysis:

Statistical analysis was performed using the student T-test. P-values<0.05 were considered significant. The leukemia, fever and healthy controls groups were classified using Ward's method, and the Euclidean distances (STATISTICA software STATISTICA, StatSoft, Inc., Tulsa, Okla.), as described in an article by Everitt B., entitled: Cluster Analysis. John. Wiley and Sons, New York (1980).

### Experimental Data

The experiments described hereinbelow were performed by the inventors in accordance with applications of the present invention and using the techniques described hereinabove.

### Example 1

In this set of experiments, FTIR methodology was used for identification and diagnosis of leukemia by analyzing biochemical changes in mononuclear cells of leukemia patients in comparison to healthy controls, in accordance with some applications of the present invention. Additionally, in order to achieve proper diagnosis of leukemia and to reduce the possibility that any biochemical changes observed by spectral analysis may result from clinical symptoms similar to leukemia, such as high level of white blood cells and fever (as described in an article by Hoffman R., et al., entitled “Hematology-Basic Principles and Practice”, 3rd Edition 2000), mononuclear cells from patients suffering from high fever with and without high level of white blood cells were compared to those of leukemia patients.

In this set of experiments, peripheral blood mononuclear cells (PBMC), from healthy controls, subjects with fever and leukemia patients (in accordance with Table I) were analyzed by FTIR-MSP, to evaluate which biochemical changes are most characteristic of mononuclear cells of leukemia patients. The PBMC was obtained by preliminary processing of the peripheral blood in accordance with the protocols described hereinabove with reference to isolation of peripheral blood mononuclear cells (PBMC). The PBMC samples were then analyzed by FTIR-MSP in accordance with the protocols described hereinabove with reference to FTIR-Microspectroscopy. It is noted that the PBMC samples for this set of experiments were obtained prior to the initiation of anti-cancer treatment, e.g., chemotherapy.

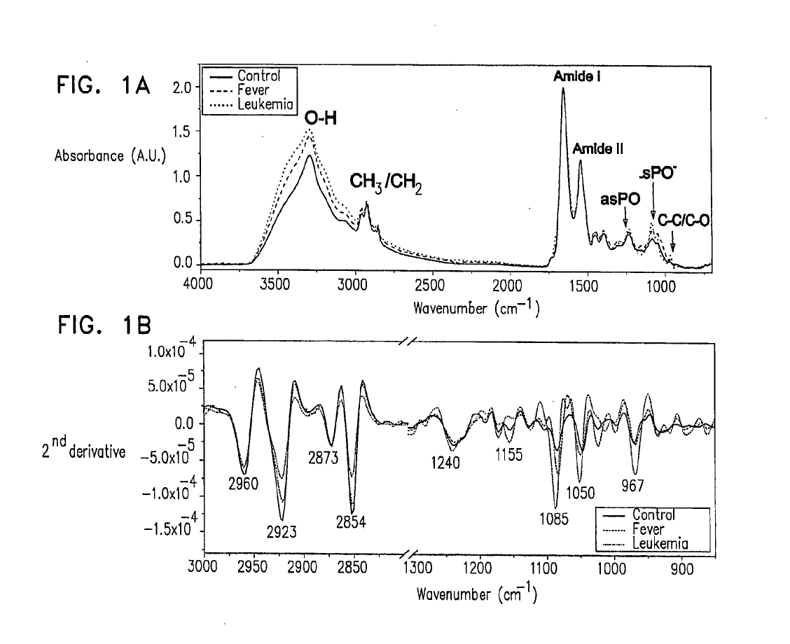

FIG. 1A shows representative FTIR-MSP spectra of mononuclear cells of healthy controls compared to FTIR-MSP spectra of mononuclear cells of leukemia patients and subjects with a fever and/or high WBC count, after baseline correction and Min-Max normalization to amide II. Each spectrum represents the average of five measurements at different sites for each sample. The spectra include a plurality of absorption bands, each corresponding to specific functional groups of specific macromolecules such as lipids, proteins, and carbohydrates/nucleic acids. The main absorption bands are marked. The FTIR spectrum was analyzed by tracking changes in absorption (intensity and/or shift) of these macromolecules.

The region 3000-2830 cm-1 contains symmetric and anti-symmetric stretching of CH3 and CH2 groups which correspond to proteins and lipids. The region 1800-1500 cm⁻¹ corresponds to amide 1 and amide II, which contain vital information regarding the secondary structures of proteins. The region 1300-900 cm-1 includes the symmetric and anti-symmetric vibrations of PO2- groups as well as other vibrations corresponding to proteins, carbohydrates, lipids and nucleic acids (as described in an article by Mantsch M and Chapman D., entitled: Infrared spectroscopy of bio molecules. John Wiley New York 1996).

As shown in FIG. 1A, the FTIR-MSP spectra derived from analysis of mononuclear cells from the leukemia patients exhibited a different spectral pattern when compared to the FTIR-MSP spectra of PBMC of healthy controls and subjects with a fever and/or high WBC count.

Reference is made to FIG. 1B. In order to increase accuracy and achieve effective comparison between leukemia, fever, and control mononuclear cells, the second derivative of the baseline-corrected, vector-normalized FTIR-MSP spectra was used. Results are presented in FIG. 1B. As shown, mononuclear cells of leukemia patients have an absorption pattern which is distinct from those of the fever and control groups.

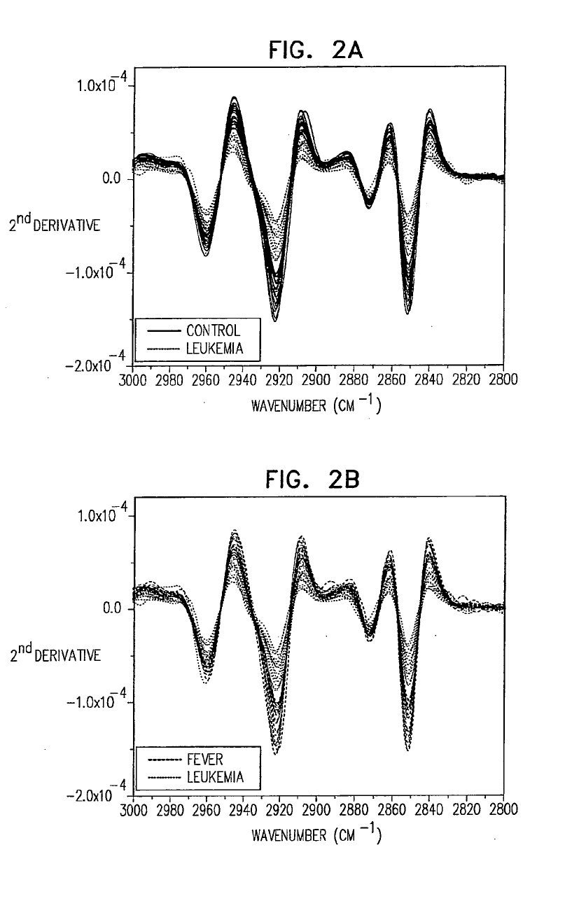

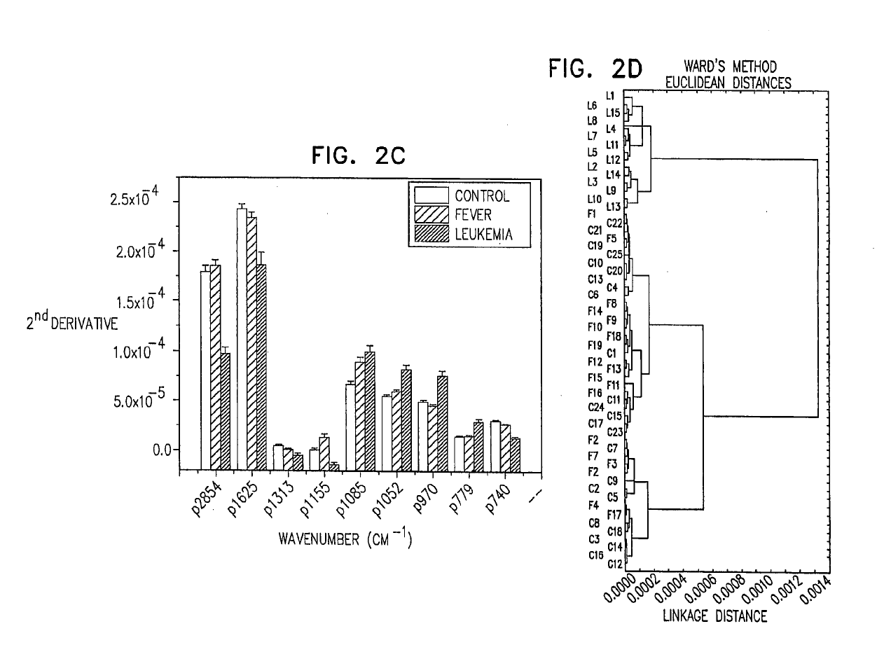

Reference is made to FIGS. 2A-D.

Reference is first made to FIGS. 2A-B. To evaluate which bands may be useful for leukemia diagnosis, further spectral analysis was conducted. FIGS. 2A-B show second derivative analysis of the IR spectra in the region 2800 to 3000 cm-1, as obtained from 15 leukemia patients, 19 fever patients and 27 healthy controls after baseline correction and vector normalization. Clear distinctive differences between the leukemia patients, subjects with fever, and healthy controls are seen in the bands corresponding to lipids and proteins in the region of 3000-2800 cm-1 as shown in FIGS. 2A-B.

Reference is now made to FIG. 2C, which is a graph representing statistical analysis of selected bands of the FTIR-MSP spectra of FIG. 1. The bands shown represent spectral changes which distinguish leukemia patients from other groups (i.e., subjects with fever and healthy controls), and are statistically significant (p<0.05). Each band corresponds to a specific functional group of different macromolecules, as listed in Table II below.

Table II represents main IR absorption bands for PBMC, and their corresponding molecular functional groups. The region 3000-2830 cm-1 contains symmetric and antisymmetric stretching of CH3 and CH2 groups, which correspond mainly to proteins and lipids respectively. The region 1700-1500 cm-1 corresponds to amide I and amide II, which contain information regarding the secondary structures of proteins. The region 1300-1000 cm-1 includes the symmetric and antisymmetric vibrations of PO2- groups. 1000-700 cm-1 is the ‘finger print’ region which contains several different vibrations corresponds to carbohydrates, lipids, nucleic acids and other bio-molecules as described in Mantsch, 1996 (referenced above). It is noted that the scope of the present invention includes the use of any suitable normalization method or any other spectral manipulation which utilizes the bands described herein, such as 966/amide 11 or CH2/CH3 at 2835-3000 cm-1 (optionally in combination with one or more other bands).

TABLE II

| Wavenumber | |
|---|---|
| (cm−1) ± 4 | Assignment |
| 2958 | νₐₛ CH₃, mostly proteins, lipids |
| 2922 | νₐₛ CH₂, mostly lipids, proteins |
| 2873 | vₛ CH₃, mostly proteins, lipids |
| 2854 | vₛ CH₂, mostly lipids, proteins |
| ~1,656 | Amide I ν C═O (80%), ν C—N (10%), δ N—H |
| ~1,546 | Amide II δ N—H (60%), ν C—N (40%) |
| 1400 | ν COO—, δ s CH3 lipids, proteins |
| 1313 | Amide III band components of proteins |
| 1240 | νₐₛ PO₂⁻, phosphodiester groups of nucleic acids |
| 1170 | C—O bands from glycomaterials and proteins |
| 1155 | νC—O of proteins and carbohydrates |
| 1085 | νs PO2− of nucleic acids, phospholipids, proteins |
| 1053 | ν C—O & δ C—O of carbohydrates |
| 996 | C—C & C—O of ribose of RNA |
| 967 | C—C & C—O of deoxyribose skeletal motions of DNA |
| 780 | sugar-phosphate Z conformation of DNA |
| 740 | ν N═H of Thymine |

Reference is now made to FIG. 2D, which represents cluster analysis according to Ward's method of the leukemia patients, the subjects with fever, and the healthy controls, in accordance with some applications of the present invention. As presented in FIG. 2D, the letters L, F and C indicate leukemia, fever and controls respectively. The diagnostic bands shown in FIG. 2C were used as inputs for the cluster analysis. These bands comprise a vector of variates for each individual subject and were thus used for cluster analysis to further evaluate the utility of FTIR-MSP for leukemia diagnosis. FIG. 2D shows a unique profile for leukemia patients, which appear as a single group, distinct from the remaining tested subjects (i.e. subjects with fever and healthy controls). However, as shown, this specific vector cannot be used to distinguish between fever patients and healthy controls, which together form a single cluster.

As shown in FIGS. 1A-B and 2A-D, PBMC of leukemia patients typically exhibit a unique FTIR spectral pattern when compared to PBMC from healthy controls or subjects with a high fever with and without a high level of white blood cells. Therefore, FTIR-MSP is shown to be an effective method for leukemia diagnosis.

### Example 2

In this set of experiments, FTIR methodology was used for monitoring of the 15 leukemia patients (in accordance with Table I) during the course of chemotherapy treatment. As provided by some applications of the present invention, FTIR methodology was used for monitoring the effect of chemotherapy treatment, by analyzing biochemical changes in PBMC of the leukemia patients. Typically, selected FTIR diagnostic bands were utilized for the monitoring of the effects of cytotoxic drugs on the mononuclear cells during chemotherapy. It is noted that any suitable wavenumbers, i.e., FTIR diagnostic bands, as described hereinabove with reference to FIGS. 1 and 2 may be used as appropriate. Optionally but not necessarily, FTIR-MSP for monitoring effects of treatment is used in combination with available common methods for assessment of Minimal Residual Disease (MRD), e.g., flow cytometry.

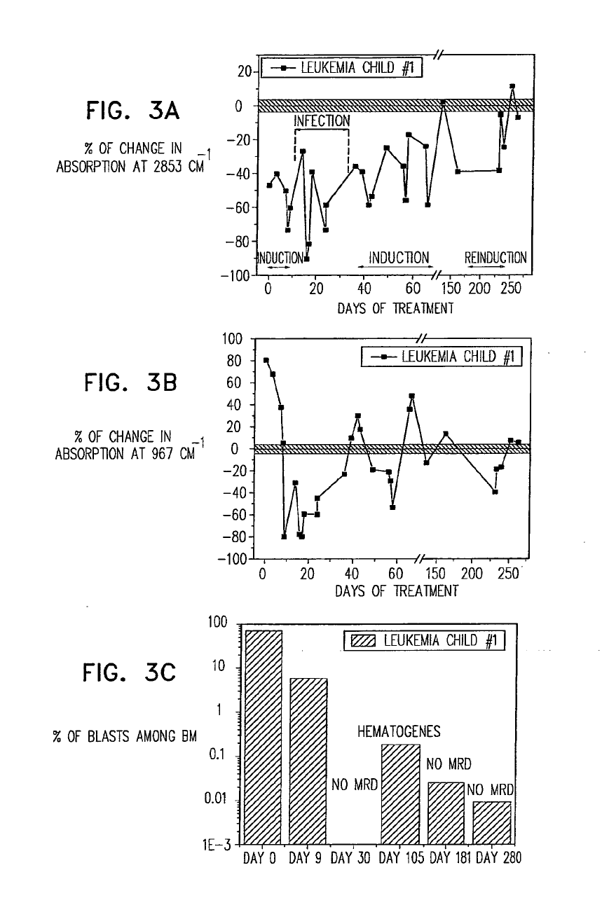

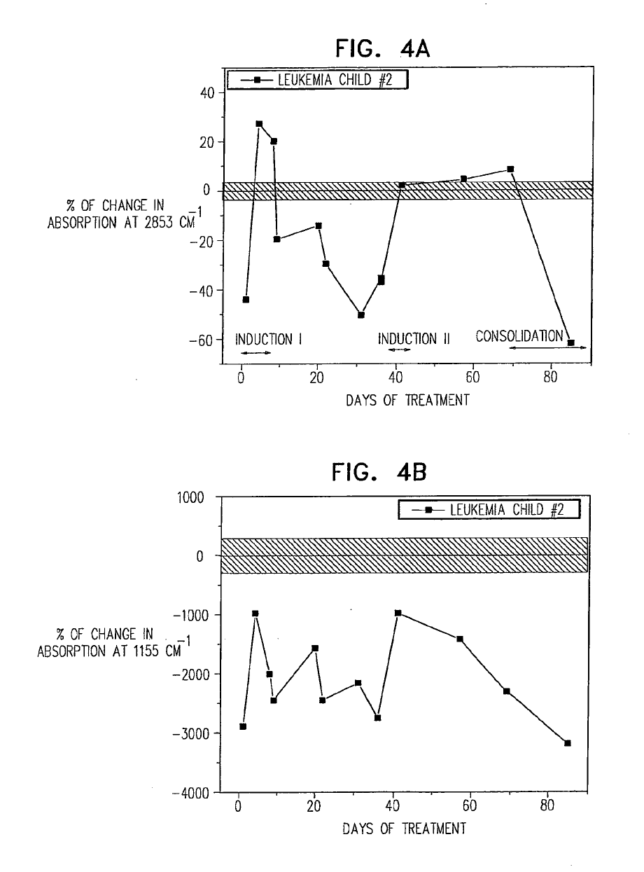

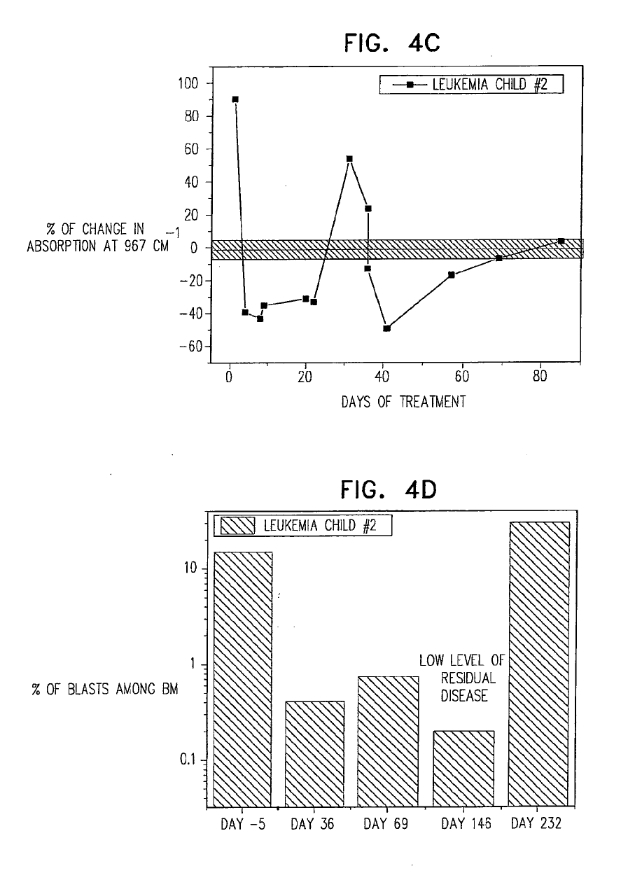

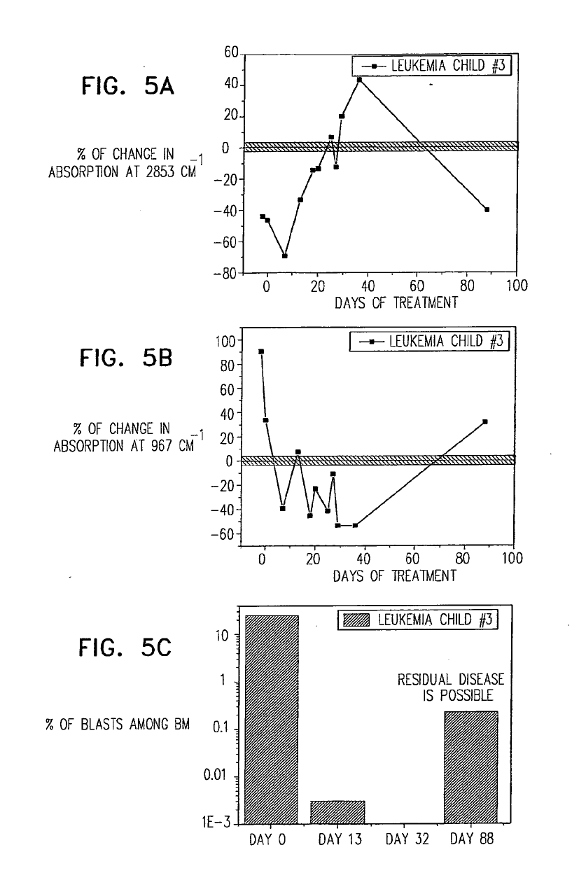

Since each patient was subjected to a different treatment protocol and presented a unique response according to the type of leukemia, described hereinbelow with reference to FIGS. 3-5 are three individual patients who responded differently to chemotherapy, representing a good prognosis (FIGS. 3A-C), an unfavorable prognosis (FIGS. 4A-D) and relapse after a short remission (FIGS. 5A-C).

Reference is made to FIGS. 3A-C, which are graphs representing FTIR-MSP spectral analysis of mononuclear cells from peripheral blood (PB), and flow cytometry analysis of blasts percentages in bone marrow (BM) samples taken from patient #1 (in accordance with Table I), during treatment.

Patient #1 is a three year old infant who was diagnosed with early B ALL. The white blood cell (WBC) count was 10,440 cells/μl, with 40% blasts in the peripheral blood (PB) and 90% blasts in the bone marrow (BM). The prognosis was good and the patient was treated according to the ALL IC-BFM 2002 protocol. Two diagnostic bands in the FTIR-MSP spectra (2853 cm-1, corresponding to lipids, and 967 cm-1 corresponding to DNA) were selected to monitor the effect of chemotherapy on PBMC. The data are presented in FIGS. 3A-B.

FIG. 3A displays the percentage of change in lipids absorption at 2853 cm-1, in comparison with the average control value (hashed region representing the average of the healthy control values and the standard deviation (SEM)). As shown in FIG. 3A, before initiating treatment (day 0), the lipid level was about 40% below the normal (control) level and a further decline was observed over the next 10 days. In the following days, there were sharp declines and increases, relative to the same average level (i.e., the spectra obtained were still abnormal, relative to spectra derived from PBMC of healthy controls). Starting on the 35th day, a steady increase towards the normal level was observed. A final steady state was only seen after about 250 days of treatment. Detailed observations made during this monitoring of this patient revealed that the child suffered from an Escherichia coli infection on the 16th day until the 28th day and that the treatment was resumed at the 45th day.

FIG. 3B displays the percentage of change in DNA absorption at 967 cm-1, in comparison with the average control value (hashed region representing the average of the healthy control values and the standard error of the mean (SEM)). As shown in FIG. 3B, there is a constant sharp decline from 80% above the normal level before treatment (day 0) down to 80% below the normal level. By day 36, the curve reached the normal level and continued to decline with the continuation of the first induction stage.

FIG. 3C shows flow cytometry analysis of bone marrow (BM) samples of leukemia patient #1 during administration of the chemotherapy treatment. As determined by fluorescence-activated cell sorting (FACS), blasts levels were below 1% after 33 days of treatment, and no MRD was observed on following days, except with cells presenting similar blasts phenotypes, such as in the case of hematogenesis.

Reference is made to FIGS. 4A-D, which are graphs representing FTIR-MSP spectral analysis of mononuclear cells from peripheral blood (PB), and flow cytometry analysis of blasts percentages in bone marrow (BM) samples taken from patient #2 (in accordance with Table I) during treatment.

Patient #2 is a 10 year old child who was diagnosed with AML-M0. The WBC count was 10,440 cells/μl, with 1% blasts in the peripheral blood (PB) and 50% blasts in the bone marrow (BM). The prognosis was unfavorable and he was treated according to a protocol which included two induction treatments; one performed on the first day and continued for a period of 8 days, and a second treatment which began on the 38th day and continued for a period of 6 days, followed by an induction period beginning on the 70th day.

As described hereinabove with reference to FIGS. 3A-B, two diagnostic bands in the FTIR-MSP spectra (2853 cm-1, corresponding to lipids, and 967 cm-1, corresponding to DNA) were selected to monitor the effect of chemotherapy on PBMC. The data regarding monitoring of patient #2 are presented in FIGS. 4A-B.

FIG. 4A displays the percentage of change in lipids absorption at 2853 cm-1, in comparison with the average control value (hashed region representing the average of the healthy control values and SEM). As shown in FIG. 4A, the lipids absorption increased on the first days beyond the normal level, followed by a decrease back to the initial level, below the control region after the first induction.

On the 30th day, there was an increase that reached stability at the normal level. However, other diagnostic bands, such as those at 1.155 cm-1, 1085 cm-1 and 740 cm-1, presented abnormal absorption values on these days (i.e., on days in which 2853 cm-1 exhibited normal absorption levels). FIG. 4B presents an abnormal absorption pattern at 1155 cm-1 exhibited during all days of treatment.

On the 85th day, the lipids level, as determined by the 2853 cm-1 diagnostic band, dropped back below the initial level.

FIG. 4C displays the percentage of change in DNA absorption at 967 cm-1, in comparison with the average control value (hashed region representing the average of the healthy control values and SEM). The changes in DNA absorption were found to correlate with the treatment days, similarly to the changes described with reference to FIG. 3B, in which a decline was observed following each induction treatment period followed by an eventual increase to the normal level. The consolidation treatment, however, is not seen to have significant influence on the DNA absorption level by the 70th day.

FIG. 4C shows flow cytometry analysis of bone marrow (BM) samples of leukemia patient #2 during administration of the chemotherapy treatment. As determined by fluorescence-activated cell sorting (FACS), although the blasts level decreased, complete remission was not established and unfortunately, following a drastic increase in blast count on day 232, this patient passed away.

Reference is made to FIGS. 5A-C, which are graphs representing FTIR-MSP spectral analysis of mononuclear cells from peripheral blood (PB), and flow cytometry analysis of blasts percentages in bone marrow (BM) samples taken from patient #3 (in accordance with Table I) during treatment.

Patient #3 is a 2 year old infant who was diagnosed with pre-B ALL. The WBC count was 7,600 cells/μl, with 13% blasts in the peripheral blood (PB) and 80% blasts in the bone marrow (BM). The prognosis was good, and the patient was treated according to the BFM 2002 protocol. As described hereinabove with reference to FIGS. 3A-B and 4A-B, two diagnostic bands in the FTIR-MSP spectra (2853 cm-1, corresponding to lipids, and 967 cm-1, corresponding to DNA) were selected to monitor the effect of chemotherapy on PBMC. The data regarding monitoring of patient #3 are presented in FIGS. 5A-B.

FIG. 5A displays the percentage of change in lipids absorption at 285.3 cm-1, in comparison with the average control value (hashed region representing the average of the healthy control values and SEM). As shown in FIG. 5A, lipid absorption declined in the first initial days of treatment and subsequently the levels rose to the normal level and beyond. However; on the 88th day, the measured lipids absorption returned to the initial pre-treatment level.

FIG. 5B displays the percentage of change in DNA absorption at 967 cm-1, in comparison with the average control value (hashed region representing the average of the healthy control values and SEM). Changes in DNA absorption were also similar to the data presented in FIGS. 3B and 4B, in which DNA absorption declined with treatment to a value below the normal level but by day 90, the DNA absorption rose above the normal level, indicating a possible relapse.

FIG. 5C shows flow cytometry analysis of bone marrow (BM) samples of leukemia patient #3 during administration of the chemotherapy treatment. As determined by fluorescence-activated cell sorting (FACS), the level of blasts declined sharply, as shown in FIG. 5C, indicating a favorable response. However, on the 88th day, there was an indication of Minimal Residual Disease (MRD), which corresponds to the lipids state returning to the initial pre-treatment level on the 88th day, as described with reference to FIG. 5A.

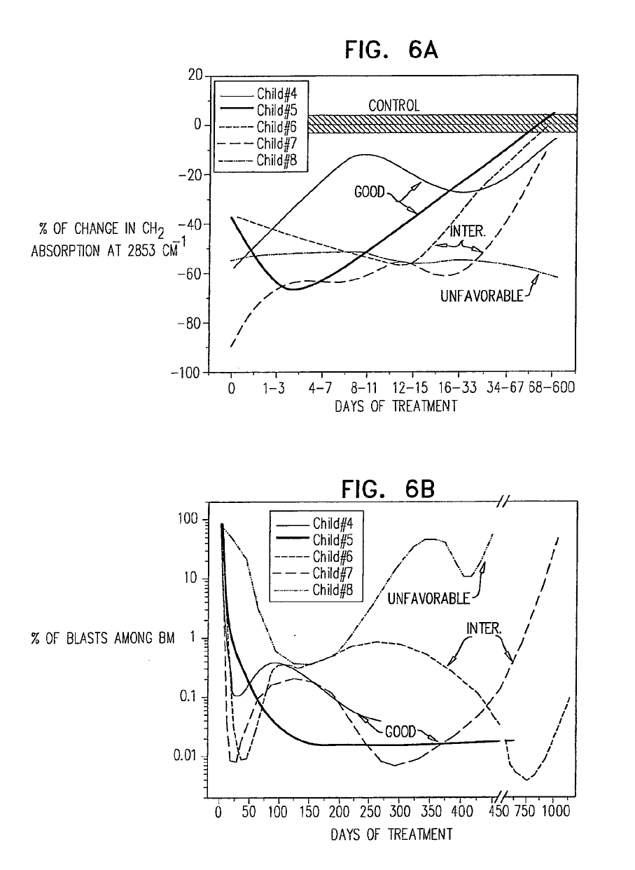

Reference is now made to FIG. 6A, which is a graph showing an additional five representative cases of leukemia patients #4-8 (in accordance with Table T), which exhibited changes in PBMC lipids during chemotherapy, as determined by FTIR-MSP. Relative absorption values were calculated from the second derivative spectra related to lipids (2853 cm-1), in comparison to healthy controls values (hashed region representing the average of the healthy control values and SEM).

As shown in FIG. 6A, FTIR spectral tendencies towards normal levels in leukemia patients undergoing treatment may be classified as good, intermediate and unfavorable responses as follows:

(a) patients having a good response to treatment, exhibiting a consistent trend towards normal values starting at day 7 of treatment, as shown with respect to child #4 and child #5;

(b) patients having an intermediate response to treatment, exhibiting a delayed decline (up to the 33rd day) following treatment and a later return to normal values, as shown with respect to child #6 and child #7; and

(c) patients having an unfavorable response to treatment by showing no tendency Inwards the normal levels throughout the treatment period, as shown with respect to child #8.

In the cases of patients #7 and #8, the patients died after a relapse of leukemia. In the case of patient #7, the measurement period did not include the days of relapse.

FIG. 6B shows percentages of blast cells in the bone marrow (BM) as determined by flow cytometry analysis. As shown, FACS analysis reveals a rapid decline in blasts percentages in the first fifty days in all cases, apart from case #8, which showed a more moderate decline. After about 450 days, the traces separate into 3 main groups of patient response, as evaluated by FACS analysis.

Reference is made to FIGS. 3-6. As shown, FTIR spectroscopy typically provides information regarding a patient's response to chemotherapy by following one or more diagnostic parameters (i.e., wavenumbers) and may identify unexpected complications as soon as they appear. For example, FTIR spectroscopy typically provides a global biochemical view which may alert the physician to sudden problems such as infections or appearance of MRD during treatment. Thus, the use of FTIR spectroscopy and microspectroscopy may improve treatment management by implementing daily follow-up procedures (which requires only a minimal blood sample of 1-2 ml) during chemotherapy, for each patient, in addition to or instead of known methods.

Reference is made to Examples 1-2 and FIGS. 1-6. It is to be noted that techniques described herein with reference to use of PBMC may be applied to any type of white blood cell (WBC). For example, analysis by FTIR-MSP techniques may be performed on any type of white blood cell, including but not limited to a total population of white blood cells (e.g., as obtained by red blood cell lysis).

Reference is made to Examples 1-2 and FIGS. 1-6. It is noted that the scope of the present invention includes the use of only one wavenumber diagnostic biomarker for detection and/or monitoring of a hematological malignancy, as well as the use of two, three, four, or more wavenumbers.

Reference is still made to Examples 1-2 and FIGS. 1-6. It is noted that, typically, diagnosis of the hematological cancer and/or monitoring of the treatment does not require calculating a ratio between two absorption bands obtained by FTIR-MSP techniques, in accordance with some applications of the present invention. For some applications, diagnosis of the hematological cancer and/or monitoring of the treatment do not require calculating any relationship relating individual ones of the bands

It is also noted that although applications of the present invention are described hereinabove with respect to spectroscopy, microspectroscopy, and particularly FTIR, the scope of the present invention includes the use of analysis techniques with data obtained by other means as well (for example, using a monochromator or an LED, at specific single wavenumbers).

Additionally, the scope of the present invention is not limited to any particular form or analysis of IR spectroscopy. For example, IR spectroscopy may include Attenuated Total Reflectance (ATR) spectroscopy techniques.

Further alternatively, the scope of the present invention is not limited to forms of IR spectroscopy and includes the use of any other suitable technique for analysis of lipid or other components in mononuclear cells, for diagnosis or monitoring of a hematological malignancy.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-11. (canceled)

12. A method for diagnosis of a hematological malignancy of a subject, the method comprising:
obtaining an infrared (IR) spectrum of a population of mononuclear cells by analyzing the population of mononuclear cells by infrared (IR) spectroscopy; and
based on one or more individual bands of the infrared spectrum, generating an output indicative of the presence of a hematological malignancy, without calculating a ratio between two of the bands.

13. The method according to claim 12, wherein generating the output comprises generating the output without calculating any relationship relating individual ones of the bands.

14. The method according to claim 12, wherein analyzing the cells by infrared (IR) spectroscopy comprises analyzing the cells by Fourier Transformed Infrared (FTIR) spectroscopy.

15. The method according to claim 14, wherein analyzing the cells by infrared (IR) spectroscopy comprises analyzing the cells by Fourier Transformed Infrared microspectroscopy (FTIR-MSP).

16. The method according to claim 12, wherein analyzing comprises assessing a characteristic of the mononuclear cell sample at a wavenumber of 967±4 cm-1.

17. The method according to claim 12, wherein analyzing comprises assessing a characteristic of the mononuclear cell sample at a wavenumber of 2853±4 cm-1.

18. (canceled)

19. The method according to claim 12, wherein analyzing comprises assessing a characteristic of the mononuclear cell sample at at least one wavenumber selected from the group consisting of: 2923±4, 1625±4, 1313±4, 1172±4, 1155±4, 1085±4, 1052±4, 780±4 and 740±4 cm⁻¹.

20. The method according to claim 19, wherein analyzing comprises assessing the characteristic at at least two wavenumbers selected from the group.

21. (canceled)

22. A method for monitoring the effect of an anti-cancer treatment on a subject undergoing anti-cancer treatment for a hematological malignancy, for use with a first population of mononuclear cells obtained from the subject prior to initiation of the treatment and a second population of mononuclear cells obtained from the subject after initiation of the treatment, the method comprising:
obtaining respective second derivatives of infrared (IR) spectra of the first and second populations of mononuclear cells, by analyzing the first and second populations of mononuclear cells by IR spectroscopy; and
based on the second derivatives of the IR spectra, generating an output indicative of the effect of the treatment.

23. The method according to claim 22, further comprising obtaining an IR spectrum of a third population of mononuclear cells obtained from the subject following termination of the treatment, by analyzing the third population of mononuclear cells by IR spectroscopy.

24. The method according to claim 22, wherein generating the output comprises generating the output without calculating any relationship relating individual ones of the bands.

25. The method according to claim 22, wherein analyzing the cells by IR spectroscopy comprises analyzing the cells by Fourier Transformed Infrared spectroscopy.

26. The method according to claim 25, wherein analyzing the cells by infrared spectroscopy comprises analyzing the cells by Fourier Transformed Infrared microspectroscopy (FTIR-MSP).

27. The method according to claim 22, wherein analyzing comprises assessing a characteristic of the mononuclear cell sample at a wavenumber of 967±4 cm-1.

28. The method according to claim 22, wherein analyzing comprises assessing a characteristic of the mononuclear cell sample at a wavenumber of 2853±4 cm⁻¹.

29. (canceled)

30. The method according to claim 28, wherein analyzing comprises assessing a characteristic of the mononuclear cell sample at at least one wavenumber selected from the group consisting of: 2923±4, 1625±4, 1313±4, 1172±4, 1155±4, 1085±4, 1052±4, 780±4 and 740±4 cm-1.

31. The method according to claim 30, wherein analyzing comprises assessing the characteristic at at least two wavenumbers selected from the group.

32. (canceled)

33. The method according to claim 30, wherein the effect of the treatment includes an effect selected from the group consisting of: a good response, an intermediate response, an unfavorable response, remission, and relapse; and
wherein generating the output indicative of the effect of the treatment comprises generating the output indicative of the effect selected from the group.

34-36. (canceled)

37. A method for diagnosis of a hematological malignancy, the method comprising:
obtaining an infrared (IR) spectrum of a population of mononuclear cells obtained from a subject suffering from a clinical symptom of a hematological malignancy, by analyzing the cells by infrared spectroscopy; and
based on the infrared (IR) spectrum, generating an output that indicates that it the IR spectrum is differentially indicative of the presence of a hematological malignancy versus the presence of a symptom selected from the group consisting of: fever and elevated white blood cell (WBC) count.

38-44. (canceled)
